SEQUENCE LISTING (8 sequences)

SEQ ID NO: 1 - protein, 9 aa, homo sapiens
  misc_feature (2)..(2): Xaa is hydroxyproline, depicted as “O” in the specification
PXGPXGPXG

SEQ ID NO: 2 - protein, 12 aa, homo sapiens
  misc_feature (11)..(11): Xaa is hydroxyproline, depicted as “O” in the specification
ITGARGLAGPXG

SEQ ID NO: 3 - protein, 12 aa, homo sapiens
  misc_feature (2)..(2): Xaa is hydroxyproline, depicted as “O” in the specification
AXGLRGGAGPXG

SEQ ID NO: 4 - protein, 30 aa, homo sapiens
  misc_feature (2)..(2): Xaa is hydroxyproline, depicted as “O” in the specification
PXGPXGPXGPXGPXGPXGPXGPXGPXGPXG

SEQ ID NO: 5 - protein, 30 aa, homo sapiens
  misc_feature (2)..(2): Xaa is hydroxyproline, depicted as “O” in the specification
PXGPXGPXGITGARGLAGPXGPXGPXGPXG

SEQ ID NO: 6 - protein, 30 aa, homo sapiens
  misc_feature (2)..(2): Xaa is hydroxyproline, depicted as “O” in the specification
PXGPXGPXGAXGLRGGAGPXGPXGPXGPXG

SEQ ID NO: 7 - protein, 29 aa, homo sapiens
HSQGTFTSDYSKYLDSRRAQDFVQWLMNT

SEQ ID NO: 8 - protein, 1029 aa, homo sapiens
  misc_feature (6)..(6): Xaa is hydroxyproline, depicted as “O” in the specification
GLAGYXGPAGPXGPXGPXGTSGHXGSXGSXGYQGPXGEXGQAGPSGPXGPXGAIGPSGPAGKDGESGRXGRXGERGLXGPXGIKGPAGIXGFXGMKGHRGFDGRNGEKGETGAXGLKGENGLXGENGAXGPMGPRGAXGERGRXGLXGAAGARGNDGARGSDGQXGPXGPXGTAGFXGSXGAKGEVGPAGSXGSNGAXGQRGEXGPQGHAGAQGPXGPXGINGSXGGKGEMGPAGIXGAXGLMGARGPXGPAGANGAXGLRGGAGEXGKNGAKGEXGPRGERGEAGIXGVXGAKGEDGKDGSXGEXGANGLXGAAGERGAXGFRGPAGPNGIXGEKGPAGERGAXGPAGPRGAAGEXGRDGVXGGXGMRGMXGSXGGXGSDGKXGPXGSQGESGRXGPXGPSGPRGQXGVMGFXGPKGNDGAXGKNGERGGXGGXGPQGPXGKNGETGPQGPXGPTGXGGDKGDTGPXGPQGLQGLXGTGGPXGENGKXGEXGPKGDAGAXGAXGGKGDAGAXGERGPXGLAGAXGLRGGAGPXGPEGGKGAAGPXGPXGAAGTXGLQGMXGERGGLGSXGPKGDKGEXGGXGADGVXGKDGPRGPTGPIGPXGPAGQXGDKGEGGAXGLXGIAGPRGSXGERGETGPXGPAGFXGAXGQNGEXGGKGERGAXGEKGEGGPXGVAGPXGGSGPAGPXGPQGVKGERGSXGGXGAAGFXGARGLXGPXGSNGNXGPXGPSGSXGKDGPXGPAGNTGAXGSXGVSGPKGDAGQXGEKGSXGAQGPXGAXGPLGIACITGARGLAGPXGMXGPRGSXGPQGVKGESGKXGANGLSGERGPXGPQGLXGLAGTAGEXGRDGNXGSDGLXGRDGSXGGKGDRGENGSXGAXGAXGHXGPXGPVGPAGKSGDRGESGPAGPAGAXGPAGSRGAXGPQGPRGDKGETGERGAAGIKGHRGFXGNXGAXGSXGPAGQQGAIGSXGPAGPRGPVGPSGPXGKDGTSGHXGPIGPXGPRGNRGERGSEGSXGHXGQXGPXGPXGAXGPC

(19) United States
(12) Patent Application Publication
(10) Pub. No.: US 2012/0058948 A1
(43) Pub. Date: Mar. 8, 2012

(54) Collagen peptides as immune modulators

(76) Inventor: Collin M. Stultz, Brookline, MA (US)

(21) Appl. No.: 12/311,876

(22) PCT Filed: Oct. 16, 2007
(86) PCT No.: PCT/US2007/022084

§ 371 Date: Sep. 29, 2010

Related U.S. Application Data
(60) Provisional application No. 60/851,959, filed on Oct. 16, 2006.

(51) Int. Cl. A61K 38/39; G01N 33/68; A61P 29/00; C12N 5/0786

(52) U.S. Cl. 514/17.2; 435/375; 435/7.24; 436/86

(57) ABSTRACT

The invention relates to collagen peptides, as well as related methods. The invention also relates to methods and products for modulating cytokine production and/or inflammation.

### RELATED APPLICATIONS

This application claims priority under 35 U.S.C. §119 from U.S. provisional application Ser. No. 60/851959, filed Oct. 16, 2006. The entire contents of which are herein incorporated by reference in their entirety.

### FIELD OF THE INVENTION

The present invention relates generally to collagen peptides and modifications thereof, as well as related methods, such as methods of modulating and detecting inflammation.

### BACKGROUND OF THE INVENTION

Activation of the innate immune response in diseases such as rheumatoid arthritis and atherosclerosis leads to the production of proinflammatory cytokines that can promote collagenolysis. While a number of studies suggest that inflammation plays a major role in initiating collagen degradation, the effect of collagen and collagen-degradation fragments on the inflammatory response is not well understood.

### SUMMARY OF THE INVENTION

It has been found that collagen peptides with certain amino acid compositions can modulate cytokine production, e.g., augment or suppress IL-1β production, from human peripheral blood monocytes. The results have wide-ranging implications for how amino acid variation in collagen affects disease and show that collagen degradation leads to the production of peptides that can modulate inflammation. A class of peptides capable of modulating inflammation is described herein.

Thus, in some aspects, the invention is directed to a composition comprising a collagen peptide that modulates cytokine production and/or inflammation. In one aspect a composition is provided that comprises a collagen peptide and a pharmaceutically acceptable carrier, wherein the collagen peptide is in an amount effective to modulate cytokine production when placed in contact with activated monocytes. In one embodiment the collagen peptide comprises a collagen fragment that lacks prolines and hydroxyprolines. In another embodiment the collagen peptide comprises a collagen fragment that contains at least one proline or hydroxyproline. In still another embodiment the collagen peptide comprises a collagen fragment that contains at least two, three, four or five prolines, hydroxyprolines or some combination of prolines and hydroxyprolines. In yet another embodiment the collagen fragment contains at least one proline and at least one hydroxyproline. In a further embodiment the collagen fragment contains at least one proline and at least two hydroxyprolines. In a further embodiment the collagen fragment contains at least one POG triplet. In yet a further embodiment the collagen fragment contains at least one POG triplet and at least one other proline or hydroxyproline. In still to another embodiment the collagen peptide further comprises one or more POG triplets flanking the N-terminus or C-terminus or both of the collagen fragment. In yet a further embodiment the collagen peptide comprises the amino acid sequence of SEQ ID NOs: 1, 2, 3, 4, 5 or 6. In still another embodiment the collagen peptide is not full-length native collagen.

In one embodiment the collagen peptide comprises a peptide of the formula:

(POG)ₙ-C-(POG)ₘ,

wherein P is proline, O is hydroxyproline, G is glycine, C is a collagen fragment that is at least 3 amino acids in length, n is 0-100, and m is 0-100, and wherein n and m are not both 0. In one embodiment the collagen fragment lacks prolines and hydroxyprolines. In another embodiment the collagen fragment contains at least one proline or hydroxyproline. In still another embodiment the collagen peptide comprises a collagen fragment that contains at least two, three, four or five prolines, hydroxyprolines or some combination of prolines and hydroxyprolines. In still another embodiment the collagen fragment contains at least one proline and at least one hydroxyproline. In a further embodiment the collagen fragment contains at least one proline and at least two hydroxyprolines. In yet another embodiment the collagen fragment contains at least one POG triplet. In yet a further embodiment the collagen fragment contains at least one POG triplet and at least one other proline or hydroxyproline.

In one embodiment the collagen peptide that inhibits cytokine production, e.g., IL-1β production, comprises a collagen fragment that is flexible (have a paucity of imino acids). In one embodiment the flexible collagen fragment adopts a partially unfolded conformation with a solvent exposed backbone. In one embodiment the fragment is one that lacks prolines and hydroxyprolines. In another embodiment the fragment is one that has no more than two prolines and hydroxyprolines in combination. In another embodiment the collagen peptide has an amino acid sequence that comprises the sequence as set forth as SEQ ID NO: 2 or 5.

In another embodiment the collagen peptide that promotes cytokine production, e.g., IL-1β production, comprises a collagen fragment that is rigid (or imino-rich). In one embodiment the rigid collagen fragment does not adopt a partially unfolded conformation with a solvent exposed backbone. In one embodiment the collagen peptide comprises a collagen fragment that contains at least three, four or five prolines, hydroxyprolines or some combination of prolines and hydroxyprolines. In another embodiment the collagen fragment contains at least one proline and at least two hydroxyprolines. In a further embodiment the collagen fragment contains at least one POG triplet and at least one other proline or hydroxyproline. In another embodiment the collagen peptide has an amino acid sequence that comprises the sequence as set forth as SEQ ID NO: 1, 3, 4 or 6. In still another embodiment the collagen peptide further comprises one or more POG triplets flanking the N-terminus or C-terminus or both of the collagen fragment.

In some embodiments the cytokine is IL-1β, IL-1α, IL-4, IL-10, IL-11, IL-12, IL-15, IL-17, IL-18, TNFα, CD40L or IFNγ. In another embodiment the cytokine is IL-1β. In some embodiments the collagen peptide inhibits IL-1β production. In other embodiments the collagen peptide promotes IL-1β production.

In some embodiments the collagen fragment is a mammalian collagen fragment. In one embodiment the mammalian collagen fragment is a human collagen fragment. In another embodiment the human collagen fragment is a fragment of human type I, II or III collagen. In one embodiment the collagen fragment is synthesized. In another embodiment the collagen fragment is derived from cleavage of a native collagen. In a further embodiment the native human collagen is a native human type II collagen. In yet a further embodiment the native human collagen is a native human type III collagen.

In some embodiments the compositions provided herein comprise an additional therapeutic agent. In one embodiment the additional therapeutic agent is an anti-inflammatory agent. In another embodiment the additional therapeutic agent is a pro-inflammatory agent.

In other aspects of the invention methods of using the collagen peptides described herein are provided. In one embodiment a method of modulating cytokine production or modulating inflammation comprising placing a composition comprising a collagen peptide in contact with cells that can produce a cytokine, such as activated monocytes, is provided. This method can be an in vitro method or it can be an in vivo method. In another embodiment a method of modulating cytokine production or inflammation in a subject comprising administering a collagen peptide composition to a subject in need thereof in an amount effective to modulate cytokine production or inflammation is provided.

In one embodiment inflammation is inhibited. In another embodiment inflammation is promoted.

In still another embodiment the subject is one with activated inflammatory cells. In a further embodiment the subject has an inflammatory disorder. In one embodiment the inflammatory disorder is rheumatoid arthritis, osteoarthritis or joint pain. In yet another embodiment the subject has had or is at risk of having a heart attack or atherosclerosis. In a further embodiment the subject has a low immune response. In yet a further embodiment the subject has or is at risk of having an infection or infectious disease.

In another embodiment the composition is delivered to activated inflammatory cells or inflammatory molecules. In one embodiment the composition is administered intravenously or into a joint space. In another embodiment the composition is not administered orally. In yet another embodiment the composition is not administered by inhalation. In still another embodiment the composition is not administered enterally. In a further embodiment the composition is not administered orally, by inhalation or enterally.

In still other aspects of the invention methods are provided whereby the ability of a collagen peptide to modulate cytokine production is determined. In one aspect a method of determining the ability of a collagen peptide to modulate cytokine production comprising placing a collagen peptide in the presence of cells that can produce cytokines and measuring the level of cytokines produced is provided. In one embodiment the ability of the collagen peptide to inhibit cytokine production is determined. In another embodiment the ability of the collagen peptide to promote cytokine production is determined. In still another embodiment the cells that can produce cytokines are one or more human peripheral blood monocytes. In a further embodiment the cells that can produce cytokines are one or more activated human peripheral blood monocytes.

In one embodiment the collagen peptide is synthesized. In another embodiment the collagen peptide is derived from cleavage of a native collagen. In one embodiment the native collagen is a native mammalian collagen. In still another embodiment the native mammalian collagen is a native human collagen. In a further embodiment the native human collagen is native human type I, II or III collagen. In yet a further embodiment the native human collagen is a native human type II collagen. In a further embodiment the native human collagen is a native human type III collagen.

Method of diagnosis or detecting are also provided. In one aspect the method is a method of detecting, comprising obtaining a sample from a subject, and detecting a collagen fragment that modulates cytokine production in the sample. In one embodiment the presence or absence of the collagen fragment in the sample is determined. In another embodiment the amount of the collagen fragment in the sample is determined.

In one embodiment the collagen fragment is a flexible collagen fragment. In one embodiment the flexible collagen fragment adopts a partially unfolded conformation with a solvent exposed backbone. In one embodiment the collagen fragment lacks prolines and hydroxyprolines. In another embodiment the collagen fragment has fewer than three prolines and hydroxyprolines in combination. In still another embodiment the collagen fragment is a rigid collagen fragment. In one embodiment the rigid collagen fragment does not adopt a partially unfolded conformation with a solvent exposed backbone. In yet another embodiment the collagen fragment contains at least one proline or hydroxyproline. In a further embodiment the collagen fragment contains at least three prolines and hydroxyprolines in some combination. In still a further embodiment the collagen fragment contains at least one POG triplet. In another embodiment the collagen fragment contains at least one POG triplet and at least one other proline or hydroxyproline. In still another embodiment the collagen fragment contains at least two or three POG triplets. In another embodiment the collagen fragment has the amino acid sequence as set forth in SEQ ID NOs: 1, 2 or 3.

In one embodiment the sample is from a subject that has or is suspected of having activated inflammatory cells. In another embodiment the sample is from a subject that has or is suspected of having an inflammatory disorder. In one embodiment the inflammatory disorder is rheumatoid arthritis, osteoarthritis or joint pain. In another embodiment the sample is from a subject that has or is suspected of having an inflamed joint. In yet another embodiment the sample if from a subject that has had or is at risk of having a heart attack or atherosclerosis. In still another embodiment the sample is from a subject that has or is suspected of having a low immune response. In a further embodiment the sample is from a subject has or is at risk of having an infection or infectious disease.

In one embodiment the sample is a body fluid or tissue sample. In another embodiment the sample is a blood, urine or plasma sample. In a further embodiment the sample is from the joint of a subject.

In one embodiment the results of the detection are used to determine the presence or absence of a disease or disorder in the subject. In another embodiment the results are compared to a predetermined value to determine the presence or absence of the disease or disorder. In another embodiment the results of the detection are used to determine the presence or absence of inflammation in a subject. In one embodiment the inflammation is joint inflammation. In another embodiment the results are compared to a predetermined value to determine the presence or absence of inflammation. In yet another embodiment the inflammation is joint inflammation.

In another aspect, forms are provided wherein a value for the amount of a collagen fragment found in a subject is listed. In one embodiment, the value is a relative value. The value can be an absolute value, in another embodiment. In still another embodiment, the form is in written or electronic form. In a further embodiment, the form can be viewed by a doctor or other medical person involved in treating a subject. In still a further embodiment, the form can be viewed by the subject. In one embodiment the form is in written form and can be sent to the subject. In another embodiment, the subject is one who has or is at risk of having any of the diseases, disorders or condition provided herein. In still another embodiment, the subject is one that would benefit from determining the presence or absence of inflammation. In a further embodiment the inflammation is joint inflammation.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, “having”, “containing”, “involving” and variations thereof herein, is meant to encompass the items listed thereafter and equivalents thereof as well as additional items.

### BRIEF DESCRIPTION OF DRAWINGS

The figures are illustrative only and are not required for enablement of the invention disclosed herein.

FIG. 1 shows the amino-acid sequence of human type III collagen using 1-letter amino-acid codes and peptides used to mimic various subsequences in type III collagen. The letter O denotes hydroxyproline. Peptide p1 models an imino-rich region of type III collagen, peptide p2 denotes a sequence adjacent to the unique matrix metalloprotease (MMP)-1 collagenases cleavage site (underlined, highlighted region) and p3 models a region near a scissile bond recognized by gelatinases (highlighted region). The initial cleavage of collagen occurs at the scissile bond adjacent to the region modeled by peptide p2, and conformational flexibility of this region is believed to enable collagenases to gain access to this site. The region adjacent to peptide p3 is not cleaved until collagen has completely unfolded; therefore, such regions are believed to be relatively rigid and have concealed scissile bonds in the native triple-helical structure. The sequences for p1, p2 and p3 are provided as SEQ ID NO: 1, 2 and 3, respectively. The complete sequence shown in the figure is provided as SEQ ID NO: 8.

FIG. 2 shows the IL-1β production in the presence of peptides p0, p1, p2 and p3.

FIG. 3 shows the IL-1β production from human peripheral blood monocytes (HPBMs) in cell culture. All points represent the mean+/− standard deviation of at least three samples. Stars, ‘*’, denote points where IL-1β concentration is significantly different than that obtained with LPS alone (Student's t-test for comparing means with unequal variances, p<0.05). FIG. 3A shows the IL-1β production in the presence of glucagon (peptide p0). FIGS. 3B-3D shows the IL-1β production in the presence of peptides p1, p2 and p3. Peptide concentrations are shown.

### DETAILED DESCRIPTION

The relationship between peptide sequence and inflammation is described herein and is based on the construction of collagen peptides that model regions of native collagen with certain amino acid compositions. With these peptides, the effect of collagen degradation products on human peripheral blood monocytes (HPBMs) in vitro was explored. More precisely, whether collagen peptides have a direct effect on IL-1β production from HPBMs was determined. “Native collagen”, as used herein, refers to any collagen as found in nature.

The choice of collagen sequences was facilitated by conformational analyses of the structure of collagen (Brodsky, B., and A. Persikov. 2005. Adv. Prot. Chem. 70:301-339; Stultz, C. M. 2002. J. Mol. Biol. 319:997-1003). Regions of collagen which contain a significant number of proline and hydroxyproline residues (i.e., imino acids) are relatively rigid, while regions that have a paucity of imino acids are relatively flexible in solution. These conformationally labile sequences within collagen can adopt a partially unfolded conformation with a solvent exposed backbone (Stultz, C. M. 2002. J. Mol. Biol. 319:997-1003; Fields, G. B. 1991. J. Theor. Biol. 153:585-602). From a teleological standpoint, if fully exposed regions led to increased IL-1β production, then chronic inflammation would ensue as these regions are, in principle, readily seen by inflammatory cells. It was postulated that conformationally labile sequences would have little effect on cytokine production, while rigid, relatively hidden, regions would have more of an effect on cytokine production.

The results described below in the Examples demonstrate that certain collagen peptides can modulate cytokine production and/or inflammation. Therefore, the invention provides such collagen peptides, compositions thereof, as well as related methods. As used herein, a “collagen peptide” is any peptide that has as or within its sequence the sequence of a collagen or a portion (fragment) thereof. The collagen can be a native collagen or a conservatively-substituted version thereof, i.e., it has the primary sequence of a naturally occurring collagen, or, alternatively, it may include one or more conservative substitutions. The collagen peptide can be, for example, a collagen fragment with a specific sequence as provided herein or it can be a collagen or fragment thereof with modifications made thereto, e.g., with POG triplets, wherein P is proline, O is hydroxyproline and G is glycine. Further details about this and other modifications are provided elsewhere herein. As used herein, “collagen fragment” is used to refer to a portion of a collagen but not the full-length polypeptide. The term is meant to encompass native collagen fragments as well as modified versions thereof. A modified version is in some embodiments a conservatively substituted version.

A “conservative amino acid substitution” is one in which the amino acid residue is replaced with an amino acid residue having a similar side chain. Families of amino acid residues having similar side chains are known in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The collagen peptides provided are those that modulate cytokine production and/or inflammation. In some instances, the collagen peptides are those that modulate cytokine production when placed in contact with activated monocytes, such as human peripheral blood monocytes. As used herein, “modulate cytokine production” refers to either an increase or decrease in the production of one or more cytokines due to the presence of a collagen peptide as compared to the production of the one or more cytokines when the collagen peptide is not present.

Cytokines include but are not limited to IL-1β, IL-1α, IL-4, IL-10, IL-11, IL-12, IL-15, IL-17, IL-18, TNFα, CD40L and IFNγ.

Assays to evaluate cytokine production are known to those of ordinary skill in the art. An example of such an assay is provided herein below in the Examples. The production of cytokines can be assessed by placing the collagen peptide in contact with any cell that has the ability of producing a cytokine. Such cells are known to those of ordinary skill in the art and include monocytes, chondrocytes, macrophages, fibroblasts, neutrophils, T-cells and B-cells, etc.

The collagen peptide may contain within its sequence the sequence of a collagen fragment that lacks prolines and hydroxyprolines. The collagen peptide may contain within its sequence the sequence of a collagen fragment that contains at least one proline or hydroxyproline. The collagen peptide may contain within its sequence the sequence of a collagen fragment with at least 1, 2, 3, 4, 5, 6, 7, 8, 9, 10, 12, 15, 17, 20, 25 prolines or hydroxyprolines or some combination of prolines and hydroxyprolines. The collagen peptide may contain within its sequence the sequence of a collagen fragment that contains at least one proline and at least one hydroxyproline. The collagen peptide may contain within its sequence the sequence of a collagen fragment with at least 1, 2, 3, 4, 5, 6, 7, 8, 9, 10, 12, 15, 17, 20, 25 prolines and at least 1, 2, 3, 4, 5, 6, 7, 8, 9, 10, 12, 15, 17, 20, 25 hydroxyprolines. The collagen peptide may contain within its sequence the sequence of a collagen fragment with at least 1, 2, 3, 4, 5, 6, 7, 8, 9, 10, 15, 20, 25 or more POG triplets. The collagen peptide may contain within its sequence the sequence of a collagen fragment with at least 1, 2, 3, 4, 5, 6, 7, 8, 9, 10, 15, 20, 25 or more POG triplets and at least one to other proline or hydroxyproline. The collagen fragment may be or include any portion of a collagen that is at least 3 amino acids in length. The collagen fragment may be or include any portion of a collagen that is at least 3, 4, 5, 6, 7, 8, 9, 10, 12, 15, 17, 20, 22, 25, 27, 30, 32, 35, 37, 40, 45, 50, 55, 60, 65, 70, 75, 80, 90, 100, 110, 125 or 150 amino acids in length. The collagen fragment may be 3, 4, 5, 6, 7, 8, 9, 10, 12, 15, 17, 20, 25, 30, 35, 40, 45, 50, 55, 60, 65, 70, 75, 80, 90, 100, 110, 125 or 150 amino acids in length.

The collagen peptide may also comprise a collagen or a fragment thereof modified by the addition of POG triplets to either end of the collagen or a fragment thereof (i.e., the N-terminus or C-terminus) or both. The POG triplets can be added in any number. For example, 1, 2, 3, 4, 5, 6, 7, 8, 9, 10, 12, 15, 17, 20, 25, 30, 35, 40, 45, 50, 55, 60, 65, 70, 75, 80, 85, 90, 95, 100, 110, 120, 150 or 200 POG triplets can be added to either end or both.

The collagen or a fragment thereof can also be modified by substituting an amino acid of a native sequence that is not proline or hydroxyproline with a proline or hydroxyproline. The collagen or a fragment thereof may be modified by 1, 2, 3, 4, 5, 6, 7, 8, 9, 10, 12 or 15 of such substitutions. The collagen or a fragment thereof can also be modified by the addition of at least one proline or hydroxyproline to the sequence of the collagen or a fragment thereof. For example, 1, 2, 3, 4, 5, 6, 7, 8, 9, 10, 12 or 15 prolines or hydroxyprolines or some combination thereof can be added to the sequence of the collagen or a fragment thereof. “Added to the sequence” refers to additions to the ends of the sequence or between two amino acids of the sequence or some combination thereof. The prolines or hydroxyprolines can be added consecutively or can be added separately or both in some combination. For instance, if 5 prolines are added, 2 can be added between two amino acids of the sequence, and the remaining 3 prolines can all be added between a different pair of amino acids. As another example, 2 prolines can be added between two amino acids, the third can be added between a different pair of amino acids, the fourth can be added between another different pair of amino acids and the fifth can be added to either end (i.e., the terminus).

The collagen peptides may also include or be of the following sequence:

(POG)ₙ-C-(POG)ₘ,

wherein P is proline, O is hydroxyproline, G is glycine, C is a collagen fragment that is at least 3 amino acids in length, n is 0-100, and m is 0-100, and wherein n and m are not both 0. C may be a collagen fragment that is at least 3, 4, 5, 6, 7, 8, 9, 10, 12, 15, 17, 20, 22, 25, 27, 30, 32, 35, 37, 40, 45, 50, 55, 60, 65, 70, 75, 80, 90, 100, 110, 125 or 150 amino acids in length.

The collagen peptide may also include the amino acid sequence of SEQ ID NOs: 1, 2, 3, 4, 5 or 6.

In some embodiments the collagen fragment is derived from native collagen. The collagen may be a native mammalian (e.g., mouse, dog, pig, rabbit, human, etc.) collagen. The collagen can be, for example, a native human type I, II or III collagen. Sequences for collagens are known to those of ordinary skill in the art and include, but are not limited, to the sequences provided by GenBank Accession Nos. CAA39142, AAA52038 and AAD13937. The collagen fragments can be obtained by cleavage of full-length collagen or a longer fragment of collagen. The collagen fragments can also be obtained by protein synthesis.

The peptides of the invention may be fusion peptides. A fusion peptide is a peptide, polypeptide or protein that is obtained by combining two distinct amino acid sequences. Typically, a partial sequence from one peptide, polypeptide or protein is linked to another heterologous peptide, polypeptide or protein, using art known techniques.

The peptides of the invention may be isolated. An isolated peptide or molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide or polysaccharide. Because an isolated molecular species of the invention may be admixed with a pharmaceutically acceptable carrier in a pharmaceutical preparation or be mixed with some of the components with which it is associated in nature, the molecular species may comprise only a small percentage by weight of the preparation. The molecular species is nonetheless substantially pure in that it has been substantially separated from the substances with which it may be associated in living systems.

The term substantially purified as used herein refers to a peptide which is substantially free of other proteins, lipids, carbohydrates or other materials with which it is naturally associated. One skilled in the art can purify peptides using standard techniques for protein purification. The substantially pure peptide will often yield a single major band on a non-reducing polyacrylamide gel. In the case of partially glycosylated peptides or those that have several start codons, there may be several bands on a non-reducing polyacrylamide gel, but these will form a distinctive pattern for that peptide. The purity of the peptide can also be determined by amino-terminal amino acid sequence analysis.

Peptide-based systems that are modulators of human immunity have a wide range of applications. For example, the peptides of the invention can be used to modulate inflammation. The peptides can be used to inhibit (i.e., reduce or eliminate) or promote inflammation. As another example, the peptides can be used to inhibit inflammation in the joints of patients with rheumatoid arthritis. For instance, SEQ ID NO: 2 has been shown to possess such useful properties, as described in the Examples section below. The collagen peptides described herein may also be used to promote inflammation. For instance, collagen peptides comprising SEQ ID NO: 1 or 3, also as described in the Examples, were found to promote IL-1β production.

The compositions of the invention are useful for modulating an inflammatory response in vitro or in vivo. The peptides may be used in vitro to study an inflammatory response, for instance. The collagen peptides as described herein may also be screened using in vitro assays to identify potent inflammatory modulators. For instance, a collagen peptide of the invention may be tested in an in vitro assay for the ability to inhibit or promote cytokine production.

Methods of determining the ability of a collagen peptide to modulate cytokine production are, therefore, provided. The methods may include the steps of placing a collagen peptide as described herein in the presence of cells that can produce one or more cytokines and measuring the levels of cytokines produced. In some examples, such cells are activated inflammatory cells. The method may be one in which the ability of the collagen peptide to inhibit the production of cytokines is determined. The method may also be one in which the ability of the collagen peptide to promote the production of cytokines is determined. The methods may include determining the production of any cytokine known to those of ordinary skill in the art. The method may be one in which the production of one cytokine can be determined or may be one in which the production of more than one cytokine is determined.

The production of cytokines can be determined with any of a number of assays known to those of ordinary skill in the art. An example of such an assay is provided below in the Examples.

The peptides of the invention can be used to modulate an inflammatory response in vivo. Inflammation is defined as the reaction of vascularized living tissue to injury. As such, inflammation is a fundamental, stereotyped complex of cytologic and chemical reactions of affected blood vessels and adjacent tissues in response to an injury or abnormal stimulation caused by a physical, chemical or biological agent. Inflammation usually leads to the accumulation of fluid and blood cells at the site of injury and is usually a healing process. However, inflammation sometimes causes harm, usually through a dysfunction of the normal progress of inflammation. Inflammatory disorders are those pertaining to, characterized by, causing, resulting from, or becoming affected by inflammation.

The collagen peptides may be used to modulate inflammation in a subject, for instance, by administering the composition to a subject in need thereof in an amount effective to modulate inflammation. A subject in need thereof is a subject in which modulation of inflammation is desirable. In some instances, a subject is in need of inhibiting inflammation. In other instances, a subject is in need of promoting inflammation.

A “subject” shall mean a human or vertebrate mammal including but not limited to a dog, cat, horse, cow, pig, sheep, goat, or primate, e.g., monkey.

A number of the peptides of the invention may be used as anti-inflammatory compounds to treat an inflammatory disorder in a subject. In one embodiment the peptide comprises a collagen fragment that is flexible. In another embodiment the peptide has an amino acid sequence comprising the sequence as set forth as SEQ ID NO: 2 or 5. The methods include administering to a subject an anti-inflammatory collagen peptide of the invention in an amount effective to treat an inflammatory disorder.

As used herein, an “inflammatory disorder” is intended to include a disease or disorder characterized by, caused by, resulting from, or becoming affected by inflammation. Examples of inflammatory disorders or disorders include, but are not limited to, acute and chronic inflammation disorders such as asthma, allergies such as allergic rhinitis, uticaria, anaphylaxis, drug sensitivity, food sensitivity and the like, cutaneous inflammation such as dermatitis, eczema, psoriasis, contact dermatitis, sunburn, aging, and the like, rheumatoid arthritis, osteoarthritis, joint pain, psoriatic arthritis, inflammatory bowel disease (Crohn's disease, ulcerative colitis), sepsis, vasculitis, and bursitis; autoimmune diseases such as Lupus, Polymyalgia, Rheumatica, Scleroderma, Wegener's granulomatosis, temporal arteritis, cryoglobulinemia, and multiple sclerosis; transplant rejection; osteoporosis; cancer, including solid tumors (e.g., lung, CNS, colon, kidney, and pancreas); Alzheimer's disease; heart attack, atherosclerosis; infectious disease such as bacterial, parasitic, fungal or viral (e.g., HIV or influenza) infections; chronic viral (e.g., Epstein-Barr, cytomegalovirus, herpes simplex virus) infection; ataxia telangiectasia, chronic obstruction pulmonary disease and chronic inflammatory bowel disease.

Thus, a number of the peptides are useful in some aspects of the invention as an anti-inflammatory agent for the treatment of a subject at risk of developing rheumatoid arthritis, osteoarthritis or joint pain, an infection with an infectious organism or a heart attack or atherosclerosis.

A subject at risk as used herein is a subject who has any risk of exposure to an infection causing pathogen, a promoter of heart disease or an inflammatory disorder. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. In regard to a subject with an infection or infectious disease who is in need of an anti-inflammatory collagen peptide, the subject has or is at risk of developing an undesirable inflammatory response. One of ordinary skill in the art will readily recognize that subjects having or who are at risk of having an infection or infectious disease may not develop or be at risk for developing an undesirable inflammatory response. In some of these instances, therefore, collagen peptides that are pro-inflammatory would be desirable for treating such subjects. In one embodiment the pro-inflammatory peptide is one that comprises a rigid collagen fragment. In another embodiment the peptide has an amino acid sequence comprising the sequence as set forth as SEQ ID NO: 1, 3, 4 or 6.

A subject at risk of developing heart disease, such as heart attack or atherosclerosis includes those subjects that have been identified as having one or more risk factors but that do not have the active disease during the peptide treatment. Subjects that are considered to be at risk of developing these diseases also include those at risk because of genetic or environmental factors.

In addition to the use of the peptides for prophylactic treatment, the invention also encompasses the use of the peptides for the treatment of a subject having a disease or disorder as described herein.

As used herein, the term treat, treated or treating refers to a prophylactic treatment which increases the resistance of a subject to development of the disease or disorder or, in other words, decreases the likelihood that the subject will develop the disease, as well as a treatment after the subject has developed the disease in order to fight the disease or prevent the disease from becoming worse.

A subject having an infection is a subject that has been exposed to an infectious pathogen and has acute or chronic detectable levels of the pathogen in the body. An infectious disease, as used herein, is a disease arising from the presence of a foreign microorganism in the body.

Viruses include, but are not limited to, enteroviruses (including, but not limited to, viruses that the family picornaviridae, such as polio virus, Coxsackie virus, echo virus), rotaviruses, adenovirus, and hepatitis virus, such as hepatitis A, B, C D and E. Specific examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papillomaviruses, polyoma viruses); Adenoviridae (most adenoviruses); Herpesviridae (herpes simplex virus (HSV) 1 and 2, varicella zoster virus, cytomegalovirus (CMV)); Poxviridae (variola viruses, vaccinia viruses, pox viruses); Iridoviridae (e.g., African swine fever virus); and other viruses acute laryngotracheobronchitis virus, Alphavirus, Kaposi's sarcoma-associated herpesvirus, Newcastle disease virus, Nipah virus, Norwalk virus, Papillomavirus, parainfluenza virus, avian influenza, SARs virus, West Nile virus.

In some aspects, the invention also intends to treat diseases in which prions are implicated in disease progression such as for example bovine spongiform encephalopathy (i.e., mad cow disease, BSE) or scrapie infection in animals, or Creutzfeldt-Jakob disease in humans.

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia and Actinomyces israelli.

Examples of infective fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease) and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

A number of the collagen peptides of the invention can also be useful for treating heart disease such as heart attack and atherosclerosis. Atherosclerosis, the most prevalent of cardiovascular diseases, is the principle cause of heart attack, stroke and vascular circulation problems. Atherosclerosis is a complex disease which involves many cell types, biochemical events and molecular factors. It is a disorder of large arteries that underlies most coronary artery disease, aortic aneurysm and arterial disease of lower extremities and is believed to play a major role in cerebrovascular disease (Libby, in “The Principles of Internal Medicine”, 15th ed., Braunward et al. (editors), Saunders, Philadelphia, Pa., 2001, pp. 1377-1382.).

A subject who has had a primary cardiovascular event is at an elevated risk of a secondary cardiovascular event. Examples of risk factors for a primary cardiovascular event include: hyperlipidemia, obesity, diabetes mellitus, hypertension, pre-hypertension, elevated level(s) of a marker of systemic inflammation, age, a family history of cardiovascular events and cigarette smoking.

As provided above, a number of the collagen peptides of the invention are also useful for treating inflammatory disorders, such as, for example, joint disorders. Joint disorders include but are not limited to joint pain, osteoarthritis and rheumatoid arthritis.

Osteoarthritis, also known as degenerative joint disease, is characterized by degeneration of articular cartilage as well as proliferation and remodeling of subchondral bone. The usual symptoms are stiffness, limitation of motion and pain. Osteoarthritis is the most common form of arthritis, and prevalence rates increase markedly with age. It has been shown that elderly patients with self-reported osteoarthritis visit doctors twice as frequently as their unaffected peers. Such patients also experience more days of restricted activity and bed confinement compared to others in their age group. In one study, the majority of symptomatic patients became significantly disabled during an 8-year follow-up period (Massardo et al., Ann Rheum Dis 48: 893 7 (1989).).

Rheumatoid arthritis (“rheumatoid arthritis” or “RA”) is a systematic inflammatory disorder that commonly affects the joints, particularly those of the hands and feet. The onset of rheumatoid arthritis can occur slowly, ranging from a few weeks to a few months, or the condition can surface rapidly in an acute manner. The classic early symptoms of rheumatoid arthritis include stiffness, tenderness, fever, subcutaneous nodules, achy joints and fatigue. The joints of the hands, feet, knees and wrists are most commonly affected, with eventual involvement of the hips, elbows and shoulders. As the joints stiffen and swell, any type of motion becomes very painful and difficult.

A number of the collagen peptides of the invention are also useful for treating an ischemic disease. An “ischemic disease or condition” as used herein refers to a condition characterized by local inflammation resulting from an interruption in the blood supply to a tissue due to a blockage or hemorrhage of the blood vessel responsible for supplying blood to the tissue such as is seen for myocardial or cerebral infarction. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue.

A number of the peptides of the invention may be used as pro-inflammatory compounds to treat a disorder in a subject in which inflammation is desirable, such subjects include those that have a low immune response. As used herein, “low immune response” refers to any lack of a desirable immune response or the need to enhance a desirable immune response. The methods can include administering to a subject a pro-inflammatory collagen peptide of the invention in an amount effective to treat a disease or disorder. Such diseases or disorders include, but are not limited to, infectious diseases as provided above.

The compositions provided can also include other agents, such as therapeutic agents or agents that modulate a particular pathological process. In addition, the collagen peptides of the present invention can be provided alone, or in combination with another agent. For example, a peptide of the present invention can be administered in combination with other known anti-inflammatory agents or pro-inflammatory agents. Known anti-inflammatory agents that may be used in the methods of the invention can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The peptides of the invention and the additional anti-inflammatory agents may be administered to the subject in the same pharmaceutical composition or in different pharmaceutical compositions (at the same time or at different times).

The other agent may be an anti-inflammatory agent, a pro-inflammatory agent or another agent useful for treating one or more symptoms or underlying mechanisms of the disorder being treated.

Anti-inflammatory agents include Alclofenac, Alclometasone Dipropionate, Algestone Acetonide, Alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Deflazacort, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Methylprednisolone Suleptanate, Momiflumate, Nabumetone, Naproxen, Naproxen Sodium, Naproxol, Nimazone, Olsalazine Sodium, Orgotein, Orpanoxin, Oxaprozin, Oxyphenbutazone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prednisone, Prifelone, Prodolic Acid, Proquazone, Proxazole, Proxazole Citrate, Rimexolone, Romazarit, Salcolex, Salnacedin, Salsalate, Salycilates, Sanguinarium Chloride, Seclazone, Sermetacin, Sudoxicam, Sulindac, Suprofen, Talmetacin, Talniflumate, Talosalate, Tebufelone, Tenidap, Tenidap Sodium, Tenoxicam, Tesicam, Tesimide, Tetrydamine, Tiopinac, Tixocortol Pivalate, Tolmetin, Tolmetin Sodium, Triclonide, Triflumidate, Zidometacin, Glucocorticoids, Zomepirac Sodium.

Pro-inflammatory agents include FGFR5 or a pro-inflammatory cytokine, such as IL-1β, IL-6, IL-8 IL-10, IL-12 or TNFα.

The other agent can also be an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod and resimiquimod.

Interferons are cytokines which are secreted by virus-infected cells as well as immune cells. Interferons include α and β-interferon. α and β-interferons are available as recombinant forms and have been used for the treatment of chronic hepatitis B and C infection. At the dosages which are effective for anti-viral therapy, interferons have severe side effects such as fever, malaise and weight loss.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine and terbinafine. Other anti-fungal agents function by breaking down chitin (e.g. chitinase) or immunosuppression (501 cream).

Therapeutic agents for the treatment of heart disease include but are not limited to anti-lipemic agents, anti-inflammatory agents, anti-thrombotic agents, fibrinolytic agents, anti-platelet agents, direct thrombin inhibitors, glycoprotein IIb/IIIa receptor inhibitors, alpha-adrenergic blockers, beta-adrenergic blockers, cyclooxygenase-2 inhibitors, angiotensin system inhibitor, anti-arrhythmics, calcium channel blockers, diuretics, inotropic agents, vasodilators, vasopressors, thiazolidinediones, cannabinoid-1 receptor blockers and/or any combination thereof.

Anti-lipemic agents are agents that reduce total cholesterol, reduce LDLC, reduce triglycerides and/or increase HDLC. Anti-lipemic agents include statins and non-statin anti-lipemic agents, and/or combinations thereof. Examples of statins include, but are not limited to, simvastatin (Zocor), lovastatin (Mevacor), pravastatin (Pravachol), fluvastatin (Lescol), atorvastatin (Lipitor), cerivastatin (Baycol), rosuvastatin (Crestor), pitivastatin. Examples of statins already approved for use in humans include atorvastatin, cerivastatin, fluvastatin, pravastatin, simvastatin and rosuvastatin.

Non-statin anti-lipemic agents include but are not limited to fibric acid derivatives (fibrates), bile acid sequestrants or resins, nicotinic acid agents, cholesterol absorption inhibitors, acyl-coenzyme A: cholesterol acyl transferase (ACAT) inhibitors, cholesteryl ester transfer protein (CETP) inhibitors, LDL receptor antagonists, farnesoid X receptor (FXR) antagonists, sterol regulatory binding protein cleavage activating protein (SCAP) activators, microsomal triglyceride transfer protein (MTP) inhibitors, squalene synthase inhibitors, and peroxisome proliferation activated receptor (PPAR) agonists.

Examples of fibric acid derivatives include but are not limited to gemfibrozil (Lopid), fenofibrate (Tricor), clofibrate (Atromid) and bezafibrate.

Examples of bile acid sequestrants or resins include but are not limited to colesevelam (WelChol), cholestyramine (Questran or Prevalite) and colestipol (Colestid), DMD-504, GT-102279, HBS-107 and S-8921.

Examples of nicotinic acid agents include but are not limited to niacin and probucol.

Examples of cholesterol absorption inhibitors include but are not limited to ezetimibe (Zetia).

Examples of ACAT inhibitors include but are not limited to Avasimibe, CI-976 (Parke Davis), CP-113818 (Pfizer), PD-138142-15 (Parke Davis) and F1394.

Examples of CETP inhibitors include but are not limited to Torcetrapib, CP-529414, CETi-1 and JTT-705.

One example of an FXR antagonist is Guggulsterone. One example of a SCAP activator is GW532 (GlaxoSmithKline).

Examples of MTP inhibitors include but are not limited to Implitapide and R-103757.

Examples of squalene synthase inhibitors include but are not limited to zaragozic acids.

Examples of PPAR agonists include but are not limited to GW-409544, GW-501516 and LY-510929.

Anti-thrombotic agents and/or fibrinolytic agents include tissue plasminogen activator (e.g., Activase, Alteplase) (catalyzes the conversion of inactive plasminogen to plasmin.

Anti-platelet agents include Clopridogrel, Sulfinpyrazone, Aspirin, Dipyridamole, Clofibrate, Pyridinol Carbamate, PGE, Glucagon, Antiserotonin drugs, Caffeine, Theophyllin Pentoxifyllin, Ticlopidine, Anagrelide.

Direct thrombin inhibitors include hirudin, hirugen, hirulog, agatroban, PPACK, thrombin aptamers.

Glycoprotein IIb/IIIa receptor Inhibitors are both antibodies and non-antibodies, and include but are not limited to ReoPro (abcixamab), lamifiban, tirofiban.

Examples of alpha-adrenergic blockers include doxazocin, prazocin, tamsulosin, and tarazosin.

Beta-adrenergic receptor blocking agents are a class of drugs that antagonize the cardiovascular effects of catecholamines in angina pectoris, hypertension and cardiac arrhythmias. Beta-adrenergic receptor blockers include, but are not limited to, atenolol, acebutolol, alprenolol, befunolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol,7-(2-hydroxy-3-t-butylaminpropoxy)phthalide. The above-identified compounds can be used as isomeric mixtures, or in their respective levorotating or dextrorotating form.

Cyclooxygenase-2 (COX-2) include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors“; U.S. Pat. No. 5,677,318 Diphenyl-1,2-3-thiadiazoles as anti-inflammatory agents”; U.S. Pat. No. 5,691,374 “Diaryl-5-oxygenated-2-(5H) -furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived.

Examples of angiotensin II receptor antagonists include but are not limited to: Candesartan (Alacand), Irbesartan (Avapro), Losartan (Cozaar), Telmisartan (Micardis), and Valsartan (Diovan). Other examples of angiotensin II antagonists include: peptidic compounds (e.g., saralasin, [(Sar¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo [4,5-c] pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl] 1H-imidazole-5-yl[methylane]-2-thiophenepropanoic acid, Smith Kline Beecham Pharmaceuticals, Pa.); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D. Searle and Company).

Angiotensin converting enzyme (ACE), inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

Calcium channel blockers are a chemically diverse class of compounds having important therapeutic value. Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Diuretics include but are not limited to carbonic anhydrase inhibitors, loop diuretics, potassium-sparing diuretics, thiazides and related diuretics.

Vasodilators include but are not limited to coronary vasodilators and peripheral vasodilators.

Vasopressors are agents that produce vasoconstriction and/or a rise in blood pressure. Vasopressors include but are not limited to dopamine, ephedrine, epinephrine, Methoxamine HCl (Vasoxyl), phenylephrine, phenylephrine HCl (Neo-Synephrine) and Metaraminol.

Thiazolidinediones include but are not limited to rosigletazone (Avandia), pioglitazone (Actos), troglitazone (Rezulin). Combination therapies of thiazolidinediones and other agents such as rosiglitazone and metformin (Avandamet) are encompassed by this invention.

One example of a cannabinoid-1 receptor blocker is rimonabant.

The peptide and other agent may be administered simultaneously or sequentially. When the peptide and other agent are administered simultaneously they can be administered in the same or separate formulations, but are administered at the same time. When administered sequentially with one another, the administration of the other agent and the peptide are temporally separated. The separation in time between the administration of these compounds may be a matter of minutes or it may be longer.

The peptides may be directly administered to the subject alone or with a carrier or delivery vehicle. Delivery vehicles or delivery devices for delivering other agents and peptides to subjects are known to those of ordinary skill in the art. The peptide and/or other agents may be administered alone (e.g., in saline or buffer) or using any delivery vehicles known in the art.

The term effective amount of a peptide refers to the amount necessary or sufficient to realize a desired biologic effect. For example, an effective amount of a peptide administered to a subject is that amount sufficient alone or in combination with another agent for an improved therapeutic outcome and/or for reducing a symptom or mechanism of the disease or disorder. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular peptide being administered the size of the subject, or the severity of the disease or condition. One of ordinary skill in the art can empirically determine the effective amount of a particular peptide and/or other agent without necessitating undue experimentation.

Subject doses of the compounds described herein for local delivery typically range from about 0.1 μg to 10 mg per administration, which depending on the application could be given daily, weekly or monthly and any other amount of time therebetween. More typically local doses range from about 10 μg to 5 mg per administration, and most typically from about 100 μg to 1 mg, with 2-4 administrations being spaced days or weeks apart. More typically, doses range from 1 μg to 10 mg per administration, and most typically 10 μg to 1 mg, with daily or weekly administrations. Subject doses of the compounds described herein for systemic or parenteral delivery are typically 5 to 10,000 times higher than the effective local dose, and more typically 10 to 1,000 times higher, and most typically 20 to 100 times higher. Doses of the compounds described herein for systemic or parenteral delivery when the peptides are administered in combination with other agents or in specialized delivery vehicles typically range from about 0.1 μg to 10 mg per administration, which depending on the application could be given daily, weekly, or monthly and any other amount of time therebetween. More typically doses for these purposes range from about 10 μg to 5 mg per administration, and most typically from about 100 μg to 1 mg, with 2-4 administrations being spaced days or weeks apart. In some embodiments, however, doses for these purposes may be used in a range of 5 to 10,000 times higher than the typical doses described above.

For any compound described herein the therapeutically effective amount can be initially determined from in vitro studies or using animal models. A therapeutically effective dose can also be determined from compounds which are known to exhibit similar pharmacological activities, such as other peptide based immune stimulants. The applied dose can be adjusted by those of skill in the art based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the peptide can be administered to a subject by any mode that delivers the peptide to the desired surface, e.g., local, systemic. For example, in some embodiments the peptide is administered so that it is delivered to a site of inflammation, such as an inflamed joint or a site of arterial inflammation. Administering the pharmaceutical composition of the present invention may be accomplished by any means known to the skilled artisan. Preferred routes of administration include but are not limited to direct local injection, oral, parenteral, intramuscular, intranasal, sublingual, intratracheal, inhalation, ocular, vaginal and rectal. In certain embodiments the composition is administered into a joint space or intravenously.

In some embodiments the composition is administered such that the composition or a portion thereof is delivered to activated inflammatory cells or inflammatory molecules, i.e., the composition is administered such that the peptide and/or other agent comes to be in the presence of the activated inflammatory cells or inflammatory molecules. In order to accomplish this, the compositions may be administered directly to a target tissue, such as a joint or they may be administered systemically in a delivery vehicle which allows delivery of the peptide to a particular target tissue.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances which increase the viscosity of the suspension, such as sodium to carboxymethyl cellulose, sorbitol, or dextran. Optionally, the suspension may also contain suitable stabilizers or agents which increase the solubility of the compounds to allow for the preparation of highly concentrated solutions.

In some embodiments the peptides and/or other agents are administered by injection into a joint. The term “joint” as used herein embraces any joint known in the medical arts, including joints of the fingers and toes, feet and hands, wrists and ankles, knees and elbows, neck, shoulders, back and hips.

In some embodiments the composition is administered orally. In other embodiments the composition is not administered orally. For oral administration, the compounds (i.e., peptides and/or other agents) can be formulated readily by combining the active compound(s) with pharmaceutically acceptable carriers well known in the art. Such carriers enable the compounds of the invention to be formulated as tablets, pills, draggers, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above compounds (i.e., peptide or other agent). The compounds may be chemically modified so that oral delivery of the compounds is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the compounds and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the compounds the location of release may be the stomach, the small intestine (the duodenum, the jejunum or the ileum) or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings or coatings which make the tablet easier to swallow. Capsules may consist of a hard shell (such as gelatin) for delivery of dry therapeutic (i.e., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compounds may be formulated (such as by liposome or microsphere encapsulation) and then further contained within an edible product, such as a refrigerated beverage containing colorants and flavoring agents.

One may dilute or increase the volume of the compounds with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the compounds together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the peptide either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Alternatively, the active compounds may be in powder form for constitution with a suitable vehicle, e.g., sterile pyrogen-free water, before use.

Also contemplated herein is pulmonary delivery. The peptide (or other agent) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-l-proteinase); Oswein et al., 1990, “Aerosolization of Proteins”, Proceedings of Symposium on Respiratory Drug Delivery H, Keystone, Colorado, March; (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the peptide (or other agent) dissolved in water at a concentration of about 0.1 to 25 mg of biologically active peptide per mL of solution. The formulation may also include a buffer and a simple sugar (e.g., for peptide stabilization and regulation of osmotic pressure). The nebulizer formulation may also contain a surfactant, to reduce or prevent surface induced aggregation of the peptide caused by atomization of the solution in forming the aerosol.

Nasal delivery of a composition of the present invention is also contemplated. Nasal delivery allows the passage of a pharmaceutical composition of the present invention to the blood stream directly after administering the product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the composition of the present invention solution into a chamber of defined volume, which chamber has an aperture dimensioned to aerosolize and aerosol formulation by forming a spray when a liquid in the chamber is compressed. The chamber is compressed to administer the composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The compositions are suitable for use in a variety of drug delivery systems. For a brief review of methods for drug delivery, see Langer, Science 249:1527-1533, 1990, which is incorporated herein by reference.

The peptides and optionally other agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions of the invention contain an effective amount of a peptide and optionally other agent included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the compositions also are capable of being commingled with the compounds of the present invention, and with each other, in a manner such that there is no interaction which would substantially impair the desired pharmaceutical efficiency. In some embodiments the pharmaceutically acceptable carrier and active agents are sterile.

Methods of detection and diagnosis are also provided. Methods for characterizing a subject's risk profile for developing a disease or disorder, or the extent of a disease or disorder, such as an inflammatory disorder, are, therefore, provided. As used herein, a subject's “risk profile” is a characterization of the likelihood that the subject will develop a disease or disorder. Based on this likelihood, a medical professional can then also determine whether or not treatment is needed or the type of treatment that is needed. The assessment of a subject's risk profile can occur once or can occur more than once. Therefore, the methods provided herein can be used to monitor a subject's risk for over time (e.g., over 2, 3, 4, 5, 6, 7, 8, 9, 10 or more time points). A subject's risk profile, therefore, can be determined on a routine basis (e.g., 1, 2, 3 or 4 times a year, every year or every 2, 3, 4, 5, 6, 7, 8, 9 or 10 years, etc.). The method for assessing risk can include the steps of determining the level of a collagen fragment in a sample obtained from the subject, comparing the level to a predetermined value, and characterizing the subject's risk of developing a disease or disorder based upon the level in comparison to the predetermined value. In some embodiments, a determination that the level is at, below or above a predetermined value is indicative of the subject's risk. In still other embodiments, the assessment is based on the comparison of the level of a collagen fragment to a predetermined value as well as a comparison of the level of at least one other marker of a disease or disorder to a predetermined value. Such markers are described in more detail below. In some embodiments, the subject for which a risk profile is determined is an apparently healthy subject.

Also provided is a method for diagnosing. As used herein, “diagnosing” refers to the determination of the presence or absence of a disease or disorder in a subject, the extent of the disease or disorder, or the determination that further testing is required in order to arrive at a final diagnosis. Such a method can include the steps of determining the level of a collagen fragment in a sample obtained from a subject, comparing the level to a predetermined value, and diagnosing the disease or disorder based upon the comparison. In some embodiments, a level at, below or above a predetermined value is indicative of whether or not the subject has the disease or disorder. The methods of diagnosis can also include determining the level of another marker of the disease or disorder and comparing the level of the other marker with a predetermined value. In some embodiments, a final diagnosis is reached through the assessment of two or more markers.

Also provided is a method for determining the prognosis of a disease or disorder. Prognosis refers to the onset, progression or regression of a disease or disorder. Prognosis includes determining the outcome of a disease or disorder. A prognostic method can include the steps of determining the level of a collagen fragment in a sample obtained from a subject, and comparing the level to a predetermined value. A level of at, below or above a predetermined value can be indicative of the prognosis. In one embodiment, the prognosis is that a positive outcome is expected. As used herein, a “positive outcome” refers to a reduction in one or more symptoms of the disease or disorder and/or an improvement in survival from the disease or disorder or the symptoms of the disease or disorder. One or more of the steps of the method can be repeated so that a subject's prognosis can be monitored over time. A method of determining prognosis can, therefore, be conducted on a routine basis (e.g., weekly, monthly, bimonthly or yearly). In some embodiments a method of prognosis can be conducted 1, 2, 3, 4 or 5 times a year. In other embodiments a method of prognosis can be conducted every 2, 3, 4 or 5 years. In some embodiments, in order to determine the prognosis, the level of another marker is also compared to a predetermined value. A final prognosis can, in some embodiments, be reached with the assessment of two or more markers.

Methods for determining a stage of a disease or disorder is also provided. The methods can, in some embodiments, involve determining the level of a collagen fragment in a sample from a subject as a determination of a stage of a disease or disorder in the subject. The result of the comparison being an indication of a stage of a disease or disorder.

The invention also embraces methods for evaluating the likelihood that a subject will benefit from treatment with a therapeutic agent or some combination of therapeutic agents as provided herein. In some embodiments the agent is an anti-inflammatory agent or pro-inflammatory agent. In other embodiments the agent is one used to treat the disease or disorder, such as an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 inhibitor, an angiotensin system inhibitor or an immunosuppressive agent. The method can, in some embodiments, involve determining the expression level of a marker in the subject, and comparing the level of the marker to a predetermined value.

The level of the marker in comparison to the predetermined value is indicative of whether the subject will benefit from treatment with said agent. In certain embodiments, the predetermined value is a plurality of predetermined marker level ranges and said comparing step comprises determining in which of said predetermined marker level ranges said subjects level falls. This method has important implications for patient treatment and also for clinical development of new therapeutics. Physicians select therapeutic regimens for patient treatment based upon the expected net benefit to the patient. The net benefit is derived from the risk to benefit ratio. The present invention permits selection of subjects who are more likely to benefit by intervention, thereby aiding the physician in selecting a therapeutic regimen. This might include using drugs with a higher risk profile where the likelihood of expected benefit has increased. Likewise, clinical investigators desire to select for clinical trials a population with a high likelihood of obtaining a net benefit. The present invention can help clinical investigators select such subjects.

Measurement of a collagen fragment alone or with other markers can be important in choosing therapy and determining the efficacy of therapy. The invention provides methods for determining whether a human subject will benefit from continued therapy or would benefit from a change in therapy. The benefit is typically, but not necessarily so, a reduction in the symptoms or in the rate of occurrence of a disease or disorder. One example of clinical usefulness includes identifying subjects who are less likely or more likely to respond to a therapy. The methods of the invention are also useful in predicting or determining that a human subject would benefit from continued therapy or would benefit from a change in therapy. Another example of clinical usefulness includes aiding clinical investigators in the selection for clinical trials of human subjects with a high likelihood of obtaining a net benefit.

As used herein, a “change in therapy” refers to an increase or decrease in the dose of the existing therapy, a switch from one therapy to another therapy, an addition of another therapy to the existing therapy, or a combination thereof. A switch from one therapy to another may involve a switch to a therapy with a high risk profile but where the likelihood of expected benefit is increased.

Also provided are methods for determining the efficacy of a therapy. The efficacy is typically the efficacy of the therapy in raising or lowering the level of a collagen fragment alone or in combination with raising or lowering the level of, for example, another marker, such as a marker of inflammation. This is sometimes also referred to as a positive response or a favorable response. Efficacy can be determined by a blood test(s) to determine whether the marker level(s) are raised or lowered or some combination thereof, as a result of therapy. Tests and methods for measuring levels in blood, especially serum samples, and for interpreting results of such tests are widely used in clinical practice today.

The invention also provides methods for deciding on the course of a therapy in a subject undergoing therapy or about to undergo therapy to reduce the symptoms or risk of a disease or disorder. Such a course of therapy is decided on the basis of the level of a collagen fragment alone or in combination with another marker, such as a marker of inflammation.

These methods have important implications for patient treatment and also for the clinical development of new therapies. It is also expected that clinical investigators now will use the present methods for determining entry criteria for human subjects in clinical trials. Health care practitioners select therapeutic regimens for treatment based upon the expected net benefit to the human subject. The net benefit is derived from the risk to benefit ratio. The present invention permits the determination of whether a human subject will benefit from continued therapy or would benefit from a change in therapy, thereby aiding the physician in selecting a therapy.

Predetermined values can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quartiles, the lowest quartile being individuals with the lowest risk and the highest quartile being individuals with the highest risk, or into taffies the lowest tertile being individuals with the lowest risk and the highest tertile being individuals with the highest risk. An important predetermined value for a marker is a value that is the average for a healthy human subject population (i.e., human subjects who have no signs and symptoms of a disease). The predetermined value will depend, of course, on the characteristics of the patient population in which the individual lies. In characterizing risk, numerous predetermined values can be established.

The predetermined value can depend upon the particular population of subjects selected. For example, an apparently healthy population will have a different ‘normal’ range than will a population of subjects which have or have had a disease or disorder. Accordingly, the predetermined values selected may take into account the category in which a subject falls. In some embodiments, the category of subjects are those that have been treated for a disease or disorder. The predetermined value, in some of these embodiments, represents a level of a marker at which or above which a therapeutic benefit has been observed in treated subjects. In other embodiments, the predetermined value represents a level of a marker at which or below which a therapeutic benefit has been observed in treated subjects. In still other embodiments, the category of subjects are those that are healthy. Appropriate ranges and categories can be selected with no more than routine experimentation by those of ordinary skill in the art.

The expression level can be determined by contacting a sample obtained from a subject with an agent that specifically binds to a nucleic acid molecule encoding a collagen fragment or the collagen fragment itself or a peptide that includes the collagen fragment and determining the interaction between the binding agent and the nucleic acid or protein. In the case where the molecule detected is a nucleic acid molecule, such determinations can be carried out via any standard nucleic acid determination assay, including the polymerase chain reaction, or assaying with labeled hybridization probes. In the case where the molecule is a protein, such determination can be carried out via any standard immunological assay using, for example, antibodies or antigen-binding fragments thereof. Other methods of specifically and quantitatively measuring proteins include, but are not limited to: mass spectroscopy-based methods such as surface enhanced laser desorption ionization (SELDI; e.g., Ciphergen ProteinChip System), non-mass spectroscopy-based methods, and immunohistochemistry-based methods such as 2-dimensional gel electrophoresis.

The level of the collagen fragment can be determined in a body fluid, such as, for example, blood, plasma, serum, lymph, saliva, urine and the like. In one embodiment, the level of the marker can be determined in a body tissue sample from a subject. In another embodiment, the sample is from a joint. The level can be determined by ELISA, or immunoassays or other conventional techniques for determining the presence of the marker. Conventional methods include sending a sample(s) of a patient's body fluid to a commercial laboratory for measurement.

The hybridization is, preferably, performed under stringent conditions and, even more preferably, under highly stringent conditions. The term “stringent conditions,” as used herein, refers to parameters with which the art is familiar. With nucleic acids, hybridization conditions are said to be stringent typically under conditions of low ionic strength and a temperature just below the melting temperature (Tₘ) of the DNA hybrid complex (typically, about 3° C. below the Tₘ of the hybrid). Higher stringency makes for a more specific correlation between the probe sequence and the target. Stringent conditions used in the hybridization of nucleic acids are well known in the art and may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. An example of “high stringency conditions” is hybridization at 65° C. in 6×SSC. Another example of high stringency conditions is hybridization at 65° C. in hybridization buffer that consists of 3.5×SSC, 0.02% Ficoll, 0.02% polyvinyl pyrolidone, 0.02% Bovine Serum Albumin, 2.5 mM NaH₂PO₄[pH7], 0.5% SDS, 2 mM EDTA. (SSC is 0.015M sodium chloride/0.15M sodium citrate, pH7; SDS is sodium dodecyl sulphate; and EDTA is ethylenediaminetetracetic acid). After hybridization, the membrane upon which the DNA is transferred is washed at 2×SSC at room temperature and then at 0.1×SSC/0.1×SDS at temperatures up to 68° C. In a further example, an alternative to the use of an aqueous hybridization solution is the use of a formamide hybridization solution. Stringent hybridization conditions can thus be achieved using, for example, a 50% formamide solution and 42° C. There are other conditions, reagents, and so forth which can be used, and would result in a similar degree of stringency. The skilled artisan will be familiar with such conditions, and thus they are not given here.

According to the invention, expression of a nucleic acid encoding a marker can be determined using different methodologies. Such methodologies include Southern and Northern blot assays using nucleic acid probes and amplification assays such as those employing PCR. As known to those skilled in the art, large probes such as 200, 250, 300 or more nucleotides are preferred for certain uses such as Southern and Northern blots, while smaller nucleic acids will be preferred for other uses such as PCR. Nucleic acids encoding a marker protein or a portion thereof also can be used to produce fusion proteins for generating antibodies or for generating immunoassay components. Likewise, such nucleic acids can be employed to produce nonfused fragments of polypeptides, useful, for example, in the preparation of antibodies, immunoassays or therapeutic applications.

As will be recognized by those skilled in the art, the size of the nucleic acid for use in the nucleic acid detection assays is between 8, 9, 10, 11 or 12 and 100 nucleotides long (e.g., 12, 13, 14, 15, 16, 17, 18, 19, 20, 21, 22, 23, 24, 25, 26, 27, 28, 29, 30, 31, 32, 35, 40, 45, 50, 55, 60, 65, 70, 75, 80, 85, 90, 95 or 100 bases) or more, up to the entire length of the marker mRNA sequence. As mentioned above, this disclosure intends to embrace each and every fragment of the sequence, beginning at the first nucleotide, the second nucleotide and so on, up to 8 nucleotides short of the end, and ending anywhere from nucleotide number 8, 9, 10 and so on for each sequence, up to the very last nucleotide. In some embodiments, it is preferable for the fragment to be unique to the nucleic acid encoding the marker.

As mentioned above, expression of a protein can be determined with, for example, antibodies or antigen-binding fragments thereof. Antibodies include polyclonal and monoclonal antibodies, prepared according to conventional methodology. Protein expression can also be determined with binding agents derived from sources other than antibody technology. For example, such binding agents can be provided by degenerate peptide libraries which can be readily prepared in solution, in immobilized form, as bacterial flagella peptide display libraries or as phage display libraries. Combinatorial libraries also can be synthesized of peptides containing one or more amino acids. Libraries further can be synthesized of peptides and non-peptide synthetic moieties.

The step of determining can be accomplished by contacting the sample with a detectable agent, such as an isolated nucleic acid molecule that selectively hybridizes under stringent conditions to a nucleic acid molecule that encodes a marker, a polypeptide, such as an antibody or antigen-binding fragment thereof, that selectively binds the marker or a fragment thereof, or a polypeptide, such as an antibody or antigen-binding fragment thereof, that selectively binds an endogenously produced antibody directed against the marker. The methods can also comprise determining expression over a period of time.

Detectable labels include fluorescent labels, chemiluminescent labels, radioactive labels and enzymes.

In some embodiments, the methods can further include the use of one or more additional tests. In some embodiments, the one or more additional tests includes the measurement of another marker of the disease or disorder in a subject. Such markers include, for example, markers of inflammation. When a level of the marker in a subject is obtained, the level, in some embodiments, can also be compared to a predetermined value.

Markers of inflammation are well-known to those of ordinary skill in the art. Such markers include cytokines and cellular adhesion molecules. Cytokines are well-known to those of ordinary skill in the art and include human interleukin 1, 2, 3, 4, 5, 6, 7, 8, 9, 10, 11, 12, 13, 14, 15, 16 and 17. Cellular adhesion molecules are well-known to those of ordinary skill in the art and include integrins, soluble intercellular adhesion molecule (sICAM-1), ICAM-3, BL-CAM, LFA-2, VCAM-1, NCAM, PECAM, fibrinogen, serum amyloid A (SAA), lipoprotein associated phospholipase A2 (LpP1A2), sCD40 ligand, myeloperoxidase, interleukin-6 (IL-6) and interleukin-8 (IL-8).

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### Examples

Inflammation plays an important role in many processes, protecting us against pathogens, and also potentially promoting disease progression. An overactive immune response has been implicated as the etiologic agent in a number of disorders including rheumatoid arthritis and atherosclerotic heart disease. These diseases share a number of processes in common including activation of innate immunity and destruction of extracellular matrix. Degradation of type II collagen in cartilage, for example, is partly responsible for the clinical severity of RA, and excessive destruction of collagen types I and III in patients with atherosclerotic heart disease promotes atherosclerotic plaque rupture and myocardial infarctions.

A number of pro-inflammatory cytokines contribute to the pathogenesis of these disorders. The proinflammatory cytokine IL-1β, can stimulate the expression of matrix metalloproteases (MMPs) in endothelial cells and smooth muscle cells—cells which play an important role in the pathogenesis of atherosclerosis. IL-1β has also been shown to promote the expression of collagenases from fibroblasts and chondrocytes, suggesting a role for this cytokine in the pathogenesis of RA as well. The inhibiting of its effects can ameloriate signs and symptoms associated with RA. MMP-1, for example, not only cleaves native collagens, but it can also deactivate IL-1β through proteolysis, and the gelatinases, MMP-2,-3, and -9 can activate IL-1β by cleaving the IL-1β precursor protein. While it has been suggested that MMPs can modulate cytokine production, few reports have shown that collagen degradation products can influence the production of cytokines, and a definitive link between collagen degradation products and the production of IL-1β has not been established. Further, while it has been demonstrated that collagen fragments are chemotactic for human monocytes in vitro, it was not known whether collagen degradation products can activate these cells.

The initial step in the degradation of interstitial collagen is cleavage at a specific site by interstitial collagenases. Subsequent unfolding of collagen monomers exposes additional scissile bonds that can then be further cleaved. Consequently, collagenase-mediated collagen degradation produces a peptide mixture containing amino-acid sequences of various sizes. As the melting temperature of collagen-like peptides, which model subsequences from mature collagen, are typically several degrees below body temperature, collagen degradation fragments likely exist as unfolded peptide chains in vivo. Although the effect of such mixtures on mammalian cells in vitro to decipher the effect of collagen fragments on inflammation and extracellular matrix degradation has been studied, such studies did not lead to precise conclusions about how the observed biological response, if any, varies as a function of sequence differences in the degradative fragments. Therefore, to explore the relationship between peptide sequence and inflammation, collagen peptides that model regions of collagen with distinct amino-acid compositions were constructed and whether such peptides would have a direct effect on IL-1β production from HPBMs was investigated.

### Materials and Methods

### Peptide Preparation

Samples (10 mg each) of peptide p1 (Lot #10055791, Genemed Synthesis Inc., San Francisco, Calif.), p2 (Lot #10059702, Genemed Synthesis Inc.) and p3 (Lot #10059701, Genemed Synthesis Inc.) were obtained with >98% purity. Each peptide was diluted with phosphate buffered saline (PBS, 1M, pH 7.2, 20012-043, Invitrogen Corp., Carlsbad, Calif.) yielding solutions of 10 mg/mL. The peptide solutions were incubated at 4° C. for 24 hours, then stored at −80° C. until required. Glucagon (G2044, Sigma-Aldrich, St. Louis, Mo.) was stored, defrosted and treated in an identical manner to p1, p2 and p3.

### Monocyte Isolation and Culture

Human peripheral blood monocytes were isolated from whole blood supplied by a healthy male as follows. A 45 mL sample was drawn using 3.2% sodium citrate vacuum tubes. Whole blood was then diluted with a 2.5 mM PBS solution (0.1M, pH 5.0, Sterile, S02131, Teknova Inc., Hollister, Calif.) to form a solution having a 1:2 ratio of blood:PBS solution. Red blood cells were subsequently removed by density gradient separation using a 1:1 ratio of Histopaque-1077 (Sigma-Aldrich) and centrifuged for 30 minutes at 400×g at 25° C. The plasma layer was removed by aspiration, and the buffy layer, comprising lymphocytes, monocytes and platelets, was collected into two 50 mL conical tubes. To remove the excess histopaque, platelets and plasma, the cells were centrifuged at 250×g for an additional 10 minutes at 4° C. The supernatant was removed and the cells resuspended in 2.5 mM PBS sodium citrate and centrifuged again. The platelet-enriched supernatant was removed after the second wash, leaving a cell pellet in each of the two tubes.

The remainder of the monocyte isolation was carried out using a MS 130-041-201 column (MACS, Miltenyi Biotec, Bergisch Gladbach, Germany) and Human Monocyte Isolation Kit II (MACS, Miltenyi Biotec) as outlined by the manufacturer. The column was washed with 1 mL of 1% heat inactivated fetal bovine serum (1% HI-FBS, USA origin, sterile-filtered, cell culture tested, hybridoma tested, F2442 Sigma-Aldrich) in a PBS sodium citrate solution. The cell pellets were combined into one tube. The cell solution was centrifuged at 300×g at 4° C. and the supematant removed before adding 30 microliters of 1% HI-FBS, 20 microliters FCR blocking agent and 20 microliters monocyte biotin-antibody cocktail per 10⁷ cells to the pellet. The tube was lightly agitated then chilled for 10 minutes at 4° C. Anti-biotin microbeads (40 microliters) and 30 microliters of additional 1% HI-FBS per 10⁷ cells were added to each tube. The tube was vortexed for three to five seconds to mix, then set to chill at 4° C. for another 15 minutes.

After the incubation at 4° C., 2 mL of 1% HI-FBS were added, and the sample was centrifuged for 10 minutes at 300×g at 25° C. to remove excess antibodies and beads. The supernatant was removed and the pellet carefully re-suspended in 500 microliters of 1% HI-FBS. This cell solution was added to the column, and no further fluid was added until the flow of solution through the column had stopped. The tube and column were washed three times with 500 microliters of 1% HI-FBS, and the fluid was collected for cell culture. Subsequent fluorescence activated cell sorter (FACS) analysis of tissue culture samples confirmed a monocyte purity of >82%. Monocytes were seeded at 5×10⁵ per well and cultured in 10% FBS RPMI 1640 and allowed to incubate at 37° C. in 5% CO₂.

HPBM Incubation with Different Peptides

Because the peptides under investigation can spontaneously form triple-helical structures in solution, the peptides were heated to 90° C. for two minutes prior to introducing them to HPBM cultures to ensure that unfolded peptides, corresponding to the collagen-degradation products, were used for the experiments. The peptide solutions were then diluted with RPMI 1640 10% FBS at 37° C., yielding a final concentration of 1 microgram/mL. Serial dilutions of the peptide samples were performed to obtain the solutions at the desired concentration. Lipopolysaccharide solution (LPS 581-0101-L002, E. coli-based, Alexis Corp., San Diego, Calif.) was diluted with 900 microliters of 37° C. 10% FBS RPMI 1640 to make 1000 microliters of a 0.1 mg/mL solution.

Cells were washed with PBS and then exposed to 3 mL of peptide solution containing either 10% FBS RPMI 1640 media, lipopolysaccharide (3.3×10⁻⁷ M) and peptides (p1 10⁻⁷ M, p2 10⁻⁷ M or p3 10⁻⁷ M) or LPS and peptides. The combination of LPS and peptides was tested at three different concentrations of each peptide, 10⁻⁷ M, 10⁻⁸ M and 10⁻⁹ M, all with the same concentration of LPS (3.3×10⁻⁷ M). Initial samples (150 microliters) were taken in triplicate from each well, and the exposure media level returned to 3 mL using the appropriate peptide solution, which had been incubating along with the cell cultures. The samples were stored at −20° C. until the tests were complete. The sampling process was repeated every four hours for 28 hours. All samples were stored at −80° C. until IL-1β assays could be performed.

### IL-1β Assay

Samples were assayed using enzyme-linked immunoabsorbent assay (ELISA) (Human Interleukin-1 beta (IL-1β) Colorimetric ELISA Kit; EH2IL1B, Endogen Searchlight, Pierce Biotechnology, Boston, Mass.), which has a sensitivity of <1 pg/mL and range of 10.2-400 pg/mL as described by the manufacturer. Standards were dissolved in ultra pure water, then diluted with 10% FBS RPMI 1640. All samples and standards were analyzed in duplicate, and the average used for the result of each plate. At least three plates were run per peptide concentration. The samples were thawed by placing them at room temperature for one hour. Thawed samples were then centrifuged at 100×g for 10 minutes prior to the ELISA assay.

50 microliters of the reconstituted standards or the test samples were added to the wells with 50 microliters of Biotinylated Antibody Reagent. The plate was gently tapped several times to mix the solutions, carefully covered with an adhesive plate cover, and the plate was incubated for three hours at 25° C. After three hours, the plate contents were discarded, and the plate was washed vigorously with wash buffer. The washing was repeated three additional times for a total of four washes prior to the addition of streptavidin-HRP solution.

After 30 minutes, the plate was washed four times with wash solution and blotted. Then, 100 microliters of TMB Substrate Solution was added to each well. The enzymatic color reaction was allowed to develop in the dark for 30 minutes with open air access. The reaction was stopped by adding 100 microliters of stop solution to each well, upon which the substrate reaction yielded a blue solution that turned yellow. The absorbance was read within 10 minutes of completion at 450 nm. Linear regression was performed on the readings from each standard, and these data were used to estimate the concentration of IL-1β from the different samples. All statistical analyses and plots were performed with MatLab (The Mathworks, Inc., Natick, Mass.).

### Results

The choice of collagen-like sequences to study was made based on a conformational analysis of collagen. It had been found that regions of collagen that contain a significant number of proline and hydroxyproline residues (i.e., the imino acids) are relatively rigid, while regions containing a paucity of imino acids are relatively flexible in solution. Molecular modeling studies also imply that conformationally labile sequences within collagen can adopt a partially unfolded conformation with a solvent-exposed backbone. From a teleological standpoint, if fully exposed regions led to increased IL-1β production, then chronic inflammation would ensue, as these regions are, in principle, readily seen by inflammatory cells. It was postulated that conformationally labile sequences would have little effect on IL-1β production in vitro, whereas sequences corresponding to rigid, relatively hidden, regions would have more profound effects.

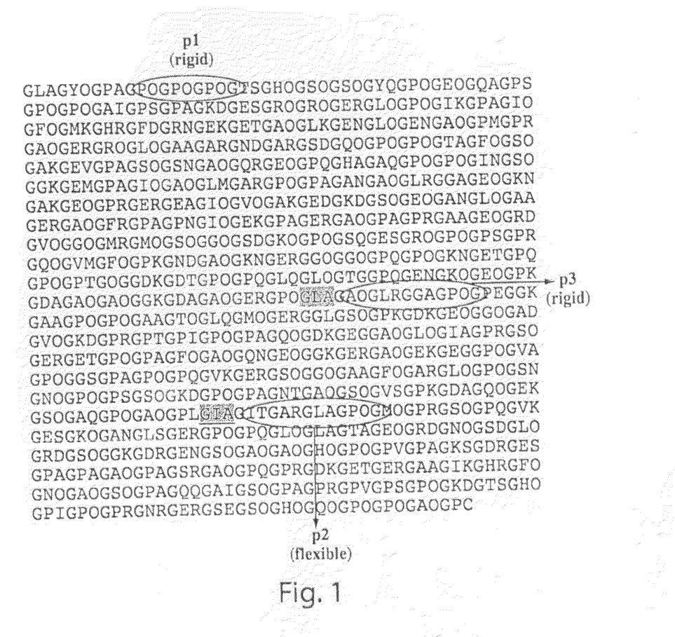

Three collagen peptides formed the basis of this work, two of which have been the focus of prior investigations on the structure of collagen (Stultz, C. M. 2002. J. Mol. Biol. 319:997-1003; Fields, G. B. 1991. J. Theor. Biol. 153:585-602; Fan, P., M. Li, B. Brodsky, and J. Baum. 1993. Biochemistry. 32:13299-13309). The first peptide, p1, consists of repeating POG triplets and, therefore, models an imino-rich segment of collagen—a region thought to adopt a rigid triple helical structure (Stultz, C. M. 2002. J. Mol. Biol. 319:997-1003). The second peptide, p2, models an imino-poor region in collagen that is thought to have considerable conformational flexibility (FIG. 1) (Stultz, C. M. 2002. J. Mol. Biol. 319:997-1003). An additional peptide, p3, was constructed, which models a relatively imino-poor segment that also represents a relatively rigid region of collagen (FIG. 1). A series of POG triplets were added to the N and C termini of the sequences, as this improves the solubility of the amino-acid sequences while helping to maintain the expected overall amino-acid composition of collagen (Table 1). These peptides were designed to model conformationally labile and conformationally rigid regions that are thought to exist in all interstitial collagens: i.e., types I, II and III.

In addition to these peptides, the effect of a non-collagenous peptide on HPBMs was tested. These data helped to determine whether any observed biological response is specific to the collagen peptides in question, or a general feature of amino-acid sequences of similar length. A literature search for naturally occurring peptides suggested that the sequence of human glucagon would be suitable (peptide p0, Table 1) (Bermudez, L. E., M. Wu, L. S. Young. 1990. Lymphokine Res. 9(2):137-45).

TABLE 1
Peptide Sequences Used in this Study

| Pep- | | |
|---|---|---|
| tide | Sequence | |
| p1 | POGPOGPOGPOGPOGPOGPOGPOGPOGPOG | SEQ ID NO: 4 |
| p2 | POGPOGPOGITGARGLAGPOGPOGPOGPOG | SEQ ID NO: 5 |
| p3 | POGPOGPOGAOGLRGGAGPAGPOGPOGPOG | SEQ ID NO: 6 |
| p0 | HSQGTIFTSDYSKYLDSRRAQDFVQWLMNT | SEQ ID NO: 7 |

In order to determine how each of the peptides affect IL-1β production in vitro, HPBMs were incubated with each peptide and IL-1β production was measured via ELISA. As collagen fragments derived from MMP-degradation are likely unfolded at body temperature, peptide samples were heated prior to incubating them with HPBMs. In order to choose an appropriate concentration, prior measurements of the concentration of collagen type II fragments in the synovial fluid from patients with RA were considered (i.e., approximately 200 ng/ml) (Elsaid, K. A., G. D. Jay, and C. O. Chichester. 2003. Osteoarthritis and Cartilage. 11(9):673-680; Elsaid, K. A., G. D. Jay, M. L. Warman, D. K. Rhee, and C. O. Chichester. 2005. Arthritis & Rheumatism. 52(6):1746-1755). Moreover, the concentration of collagen fragments in the synovial fluid of normal individuals, without RA, is below the level of detection (Elsaid, K. A., G. D. Jay, and C. O. Chichester. 2003. Osteoarthritis and Cartilage. 11(9):673-680; Elsaid, K. A., G. D. Jay, M. L. Warman, D. K. Rhee, and C. O. Chichester. 2005. Arthritis & Rheumatism. 52(6):1746-1755). Therefore, HPBMs were incubated with the peptides described above at concentration of 10⁻⁷ M (˜270 ng/ml), 10⁻⁸M (˜27 ng/ml) and below 10⁻⁹ M (˜2.7 ng/ml).

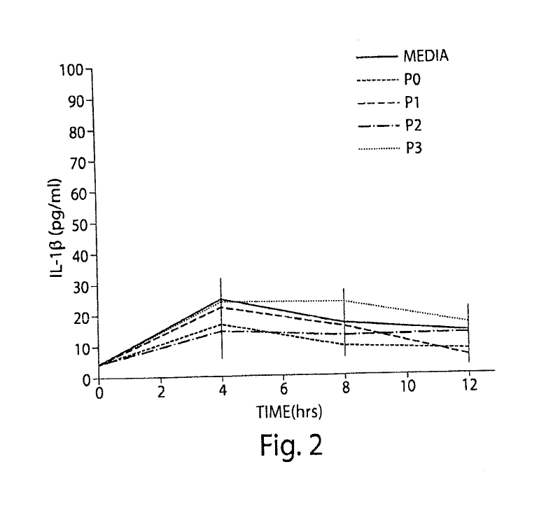

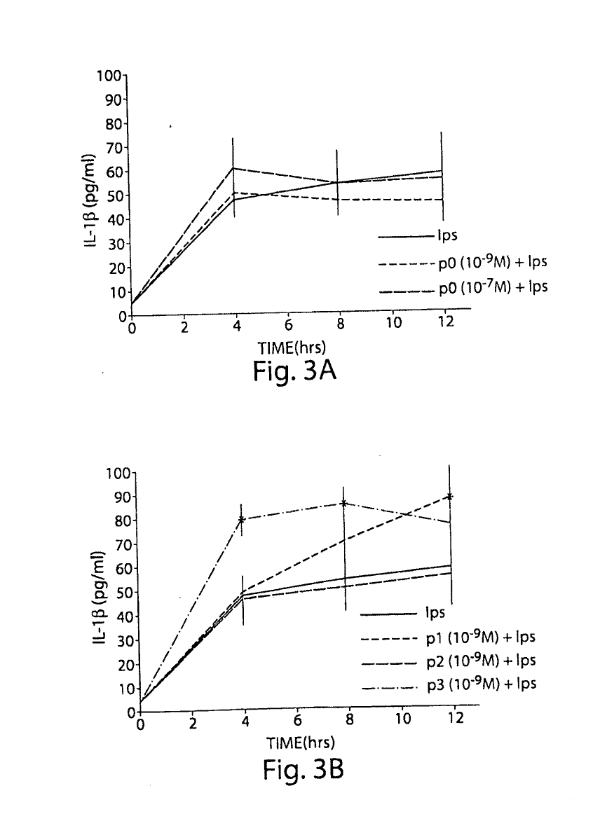

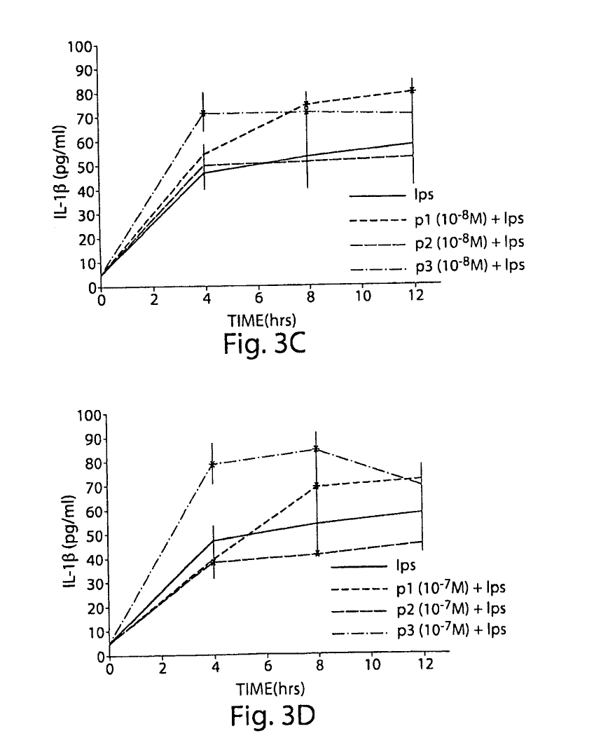

Peptides p0 to p3 had no significant affect on IL-1β production when incubated with HPBMs alone, suggesting that these peptides do not activate the innate immune response by themselves (FIG. 2). However, as patients with diseases like RA may have inappropriate activation of innate immunity leading to tissue destruction, lipopolysaccharide (LPS), a potent activator of the innate immune response, was used to determine the effect of each peptide on activated monocytes (Smolen, J. S., K. Redlich, J. Zwerina, D. Aletaha, G. Steiner, and G. Schett. 2005. Clinical Reviews in Allergy and Immunology. 28(3):239-248; Abramson, S. B., and A. Amin. 2002. Rheumatology. 41:972-980; Heumann, D., and T. Roger. 2002. Clinica Chimica Acta. 323:59-72). HPBMs incubated with LPS alone produced elevated levels of IL-1β (FIG. 3A). The addition of the non-collagenous peptide, p0, had no significant effect on IL-1β production (FIG. 3A). Although some augmentation of IL-1β levels is observed when a relatively high concentration of peptide p0 is used, this increase, however, is not statistically significant. By contrast, there was an additive effect when either peptide p1 or p3 was incubated with HPBMs (FIG. 3B). While augmentation is observed with both peptides, the greatest effect is associated with peptide p3 as elevated IL-1β levels in the presence of peptide p1 began at a later time relative to that seen with peptide p3.

Peptide p2, however, did not augment IL-1β production (FIGS. 3B and 3C). Moreover, at a relatively high concentration, peptide p2 suppressed LPS-induced IL-1β production, while IL-1β production was still augmented by the other peptides (FIG. 3D). Interestingly, although peptides p2 and p3 differ by 4 amino acids (Table 1), they have different effects on LPS-mediated IL-1β production.

### Discussion

Inflammatory mediators play an important role in many disorders of collagen metabolism, and cytokines have been suggested to affect the progression of such diseases. However, the effect of structural proteins, such as collagen, and their degradation products, on inflammation in general has not been thoroughly investigated.

Structural proteins such as collagen impart tensile strength and durability to tissues, yet there is increasing evidence that these proteins also can affect a number of physiologic processes. The results above demonstrate that collagen fragments can be either pro-inflammatory or anti-inflammatory depending on their amino acid sequence. While LPS-induced IL-1β production is augmented in the presence of p1 and p3, Il-1β is reduced in the presence of p2. The difference in amino acid sequence between peptides p2 and p3 suggests that amino acid variation in collagen fragments can lead to a different effect on the inflammatory response.

Peptides p1 and p3 augment IL-1β production at all concentrations studied (FIGS. 3B-D), suggesting that this response saturates at low peptide concentrations (FIG. 3B). By contrast, p2 suppresses IL-1β at concentrations close to that observed in the synovial fluid of patients with active RA (FIG. 3D). This finding implies that the suppressive effect of peptides, such as p2, is exerted when there is excessive cartilage destruction. As such, the effect of immunosuppressive peptides, such as p2, has a significant effect when there is excessive cartilage destruction, thereby providing a mechanism for limiting the extent of joint inflammation.

This study shows that collagen peptides can augment the production of IL-1β from human monocytes at concentrations similar to that seen in the synovial fluid of patients with RA. LPS was used to activate HPBMs in vitro, and even though it is not known whether this mode of monocyte activation is similar to the activation mechanism of monocytes in different disease states (Smolen, J. S., K. Redlich, J. Zwerina, D. Aletaha, G. Steiner, and G. Schett. 2005. Clinical Reviews in Allergy and Immunology. 28(3):239-248; Young, J. L., P. Libby, and U. Schonbeck. 2002. Thrombosis and Haemostasis. 88:554-567), LPS has been used in numerous studies to model inflammation in general. Useful insights into different diseases have been obtained from such studies (e.g., De Bont, N., M. G. Netea, C. Rovers, T. Smilde, A. Hijmans, P. N. M. Demacker, J. W. M. Van der Meer, and A. F. H. Stalenhoef. 2006. J. Interferon & Cytokine Research. 26(2):101-107; Ashida, K., K. Miyazaki, E. Takayama, H. Tsujimoto, M. Ayaori, T. Yakushiji, N. Iwamoto, A. Yonemura, K. Isoda, H. Mochizuki, H. Hiraide, M. Kusuhara, and F. Ohsuzu. 2005. J. Atherosclerosis & Thrombosis. 12(1):53-60; Kobayashi, H., M. Takeno, T. Saito, Y. Takeda, Y. Kirino, K. Noyori, T. Hayashi, A. Ueda, Y. Ishigatsubo. 2006. Arthritis & Rheumatism. 54(4):1132-1142; Inoue, T., D. L. Boyle, M. Corr, D. Hammaker, R. J. Davis, R. A. Flavell, and G. S. Firestein. 2006. PNAS. 103(14):5484-5489).

The data also showed that some collagen peptides can suppress the production of IL-1β. As mentioned previously, collagenolysis typically leads to the production of a variety of peptides having a range of different molecular weights (Vankemmelbeke, M., P. M. Dekeyser, A. P. Hollander, D. J. Buttle and J. Demester. 1998. Biochem J. 330:633-640; Eckhardt, A., I. Mikŝik, J. Charvátová, Z. Deyl, E. Forgács, and T. Cserháti. 2005. Journal of Liquid Chromatography & Related Technologies. 28:1437-1451). The results demonstrate that different peptides within this mixture have distinct effects on the production of IL-1β. Whether the mixture of collagen fragments that results from collagenolysis enhances or suppresses inflammation will depend on the identities of the produced peptides. Hence inter-patient variation in the composition of collagen fragments in the synovium, for example, may lead to different levels of inflammation.

As the peptides for this study were chosen based on a conformational analysis of collagen, the findings demonstrate that interesting biological insights can be derived from fundamental structural analyses of collagen. The results show that collagenolysis leads to the production of peptides, which can affect the inflammatory cascade by modulating cytokine production from human monocytes. Collagen fragments can be either pro-inflammatory or anti-inflammatory depending on their amino acid sequence. Consequently, the extent of joint inflammation in patients with RA, for example, can depend on whether collagenolysis leads to a preponderance of inflammatory or anti-inflammatory peptides. Therefore, in addition to being an important structural protein, collagen is now shown to be an active player in inflammatory processes that impact disease.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The citation of a reference herein is not intended to be an admission that the reference is a prior art reference.

What is claimed is:

1. A composition, comprising:
a collagen peptide and a pharmaceutically acceptable carrier, wherein the collagen peptide is in an amount effective to modulate cytokine production when placed in contact with activated monocytes.

2. The composition of claim 1, wherein the cytokine is IL-1β, IL-1α, IL-4, IL-10, IL-11, IL-12, IL-15, IL-17, IL-18, TNFα, CD40L or IFNγ.

3. The composition of claim 2, wherein the cytokine is IL-1β.

4. The composition of claim 3, wherein the collagen peptide inhibits IL-1β production.

5. The composition of claim 3, wherein the collagen peptide promotes IL-1β production.

6. The composition of claim 1, wherein the collagen peptide comprises a collagen fragment that lacks prolines and hydroxyprolines.

7. The composition of claim 1, wherein the collagen peptide comprises a collagen fragment that has fewer than three prolines and hydroxyprolines in combination.

8. The composition of claim 1, wherein the collagen peptide comprises a collagen fragment that contains at least three prolines and hydroxyprolines in some combination.

9. The composition of claim 1, wherein the collagen peptide comprises a collagen fragment that contains at least one POG triplet and at least one other proline or hydroxyproline.

10. The composition of claim 8, wherein the collagen peptide further comprises one or more POG triplets flanking the N-terminus or C-terminus or both of the collagen fragment.

11. The composition of claim 1, wherein the collagen peptide is a peptide of the formula:
(POG)ₙ-C-(POG)ₘ,
wherein P is proline, O is hydroxyproline, G is glycine, C is a collagen fragment that is at least 3 amino acids in length, n is 0-100, and m is 0-100, and wherein n and m are not both 0.

12. The composition of claim 11, wherein the collagen fragment lacks prolines and hydroxyprolines.

13. The composition of claim 11, wherein the collagen peptide comprises a collagen fragment that has fewer than three prolines and hydroxyprolines in combination.

14. The composition of claim 11, wherein the collagen fragment contains at least one proline or hydroxyproline.

15. The composition of claim 11, wherein the collagen peptide comprises a collagen fragment that contains at least three prolines and hydroxyprolines in some combination.

16. The composition of claim 11, wherein the collagen peptide comprises a collagen fragment that contains at least one POG triplet and at least one other proline or hydroxyproline.

17-25. (canceled)

26. A method of modulating cytokine production, comprising:
contacting cells that can produce a cytokine with the composition of claim 1 in an amount effective to modulate cytokine production.

27-32. (canceled)

33. A method of modulating inflammation, comprising:
contacting cells that can produce a cytokine with the composition of claim 1 in an amount effective to modulate inflammation.

34-47. (canceled)

48. A method of determining the ability of a collagen peptide to modulate cytokine production, comprising:
placing a collagen peptide in the presence of cells that can produce cytokines, and
measuring the level of cytokines produced.

49-74. (canceled)

75. A method of detecting, comprising:
obtaining a sample from a subject, and
detecting a collagen fragment that modulates cytokine production in the sample.

76-100. (canceled)
